(19) United States
(12) Patent Application Publication
(10) Pub. No.: US 2023/0404910 A1
(43) Pub. Date: Dec. 21, 2023

(54) IMPLANTABLE CANNABIS TRANSDERMAL

(71) Applicant: Stephanie Gore, Bronx, NY (US)

(72) Inventor: Stephanie Gore, Bronx, NY (US)

(21) Appl. No.: 17/842,271

(22) Filed: Jun. 16, 2022

(51) Int. Cl. A61K 9/00; A61K 31/05; A61K 31/185

(52) U.S. Cl. CPC A61K 9/0036; A61K 31/05; A61K 31/185

(57) ABSTRACT

An implantable drug delivery device for the treatment of chronic pelvic pain in human females. A non-biodegradable drug-eluting positionally adjustable silicone ring for removable placement in the cervix to provide a point of use application of pain reducing cannabis compounds in a safe and proven delivery methodology of reduced systematic exposure.

### BACKGROUND OF THE INVENTION

### 1. Technical Field

This invention relates to implantable delivery devices for the application of medication over a determined time period by use of polymetric devices of biodegradable or non-biodegradable properties.

### 2. Description of Prior Art

Prior art medication treatment systems and devices have been developed to provide a variety of transdermal dissemination of cannabidiol (CBU) using for example reservoir patch designs with a micro-porous hydrophilic membrane as seen in U.S. Pat. No. 10,588,869 which is adhesively attached to the patient's skin.

Another example of cannabidiol patch and method for transdermal delivery is evident in U.S. Pat. No. 6,113,940 wherein cannabis chemicals are contained in a film of the body layer or within a cavity between layers.

An adjustable medication delivery amount device can be seen in U.S. Pat. No. 9,033,910 having a transdermal drug delivery bracelet wherein the hollow bracelet is fitted with treatment substances using a filter to regulate dispersion.

Implantable drug delivery systems are well known in the art which by definition provides site specific drug delivery for a variety of treatment such as diabetes, contraception, chronic pain, cardiology, oncology, and central nervous system.

In women's health there are a variety of applications such as transmucosal hormone contraception, delivery progestin, or estrogen within delivery products made of silicone, EVA and TPU. An example of such as a silicone intravaginal ring (IVR) that releases 2 mg. of estradiol for 90 days for the treatment of symptoms associated with menopause.

### SUMMARY OF THE INVENTION

An implantable placement device for women's health that provides a medication release vehicle for cannabis-based ingredients for the reduction of chronic pain. The device comprises a size adjustable ring of medical grade non-biodegradable implants of silicone which are permeable and filled with cannabis indicia strain or a blend of cannabis indicia and Cannabis sativa. The device is of an adjustable ring configuration for interengagement within itself designed to fit around the cervix of the user for direct release into the pelvis.

### DESCRIPTION OF THE DRAWINGS

FIG. 1 is a front elevational view of the dispensing ring of the invention in open pre-insertion position.

FIG. 2 is an enlarged partial cross-sectional view of the inner engagement portion of the ring.

FIG. 3 is a composite view in solid and broken lines showing size adjustable ability of the ring system.

FIG. 4 is a front elevational view of the ring in closed reduced diameter position.

FIG. 5 is a top plan view on lines 5-5 of FIG. 1.

FIG. 6 is an end elevational view on lines 6-6 of FIG. 4.

FIG. 7 is a perspective view of the dispensing ring in closed position.

### DETAILED DESCRIPTION OF THE INVENTION

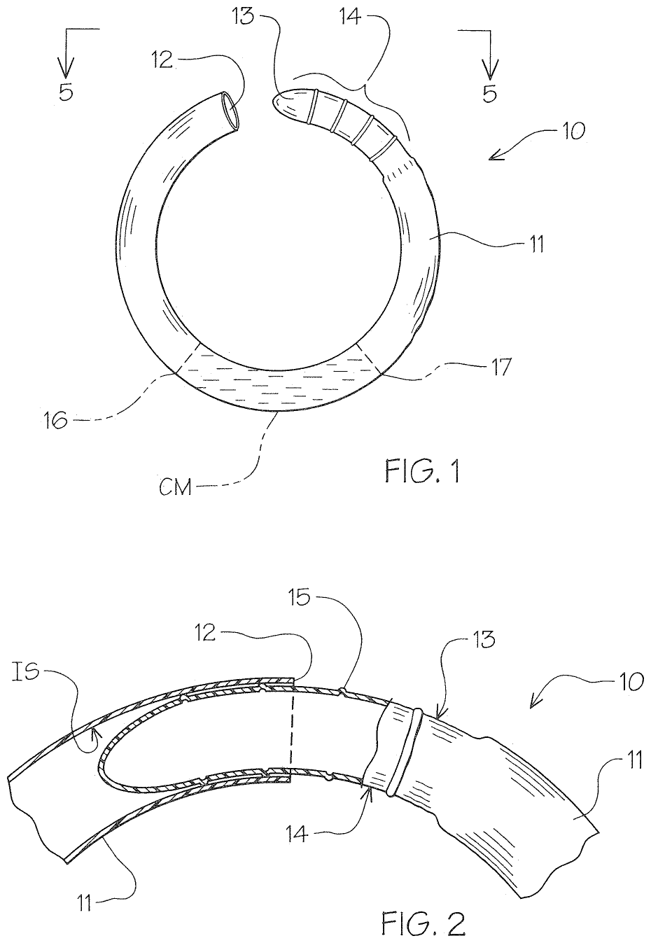

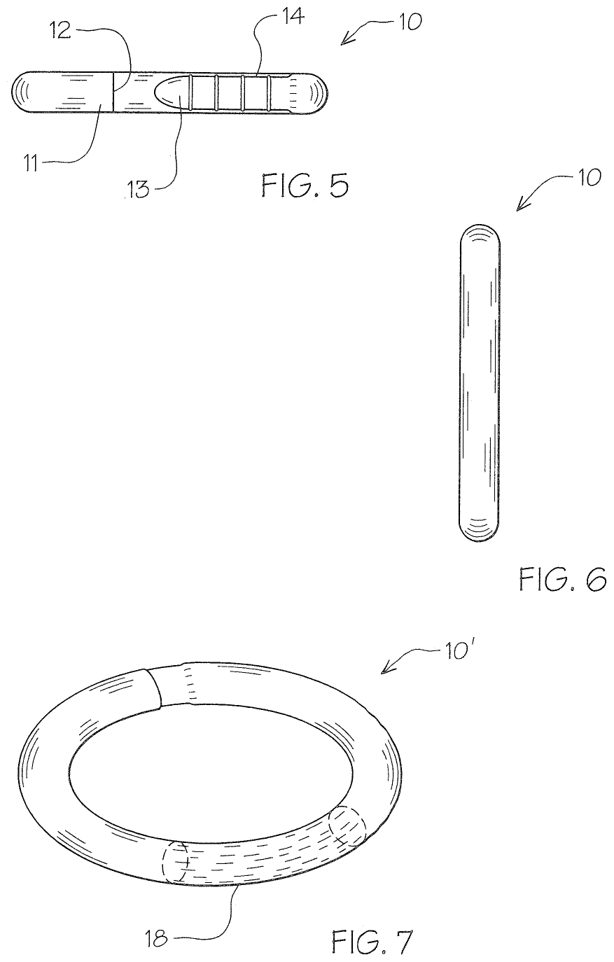

Referring to FIGS. 1-7 of the drawings, an implantable dispensing ring 10 of the invention can be seen having a hollow tubular annular main body member 11 with an open end at 12 and an affacing tapered registration end 13. The surface of the ring's main body member 11 extending from the tapered registered end 13 has an area of reduced annular dimensions 14 with a plurality of longitudinal spaced annular ribs 15 which allows for the variable foreshortening of the ring 10 upon end insertion within the aligned ring open end 12 as best seen in FIGS. 4 and 7 of the drawings.

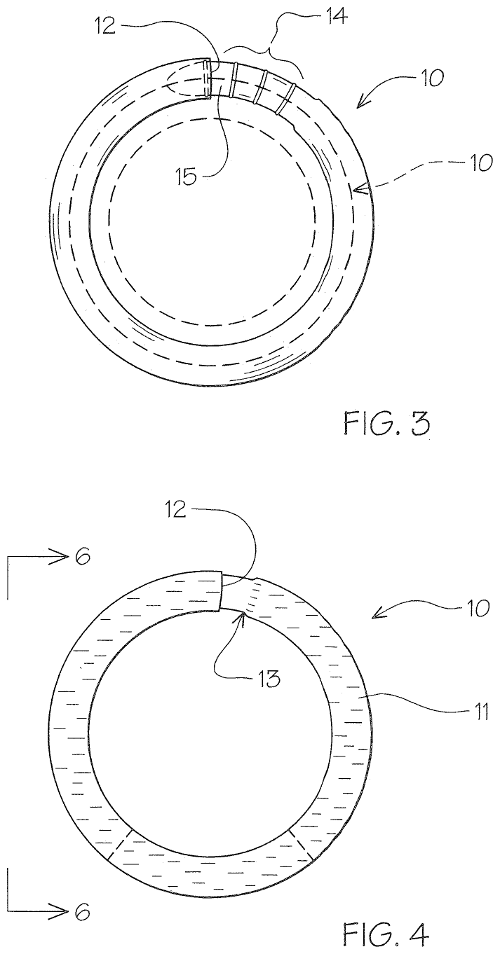

The annular engagement ribs 15 are spaced longitudinally to provide for variable sizing of the overall annular dimension of the implantable dispensing ring 10 to accommodate variation in sizing, shown in FIG. 3 of the drawings by solid and broken lines. required for different patients, not shown. The ribs 15 provide for frictional engagement on the interior ring surfaces when inserted in the open-end ring 12 imparting adjustability, as noted, to ring size as hereinbefore described.

The body of the implantable dispensing ring 10 is preferably formed of non-biodegradable medical grade silicone having inherent flexibility and yielding properties to accommodate the respective end to end insertion and retainment thereby so as to fit adjustably around the cervix of the patient upon insertion for required effective placement.

The main body member 11 of the ring 10 as hereinbefore described is of a hollow tubular construction which will provide for placement of a cannabis-based pain reduction compound CM shown graphically in FIG. 1 of the drawings within indicated by selective broken lines at 16 and 17 midway from its respective adjacent joining ends 12 and 13. It will be evident that a variety of placement protocols can be used, if required.

The ring's main body member 11 will therefore be of a permeable silicone for drug eluting properties having a known diffusion rate determine by the material chosen and wall thickness which is well known and available within the art.

Referring now to FIG. 7 of the drawings, an implantable ring 10 is shown in closed position having an alternate material construction wherein only a specific section of the body member indicated at 18 has high permeable properties for degree transfer that will correspond, in this example, to the preferred placement of the cannabis-based pain reduction compound CM therein indicated.

It will be evident from the above description that a number of treatment applications and advantages are achieved wherein the implantable dispensing ring 10 can be easily filled with the hereinbefore described cannabis compound CM chosen from a group consisting of, but not limited to cannabis, cannabis plus anti-inflammatory agent, cannabis plus progesterone, cannabis plus progesterone and estrogen, cannabis plus gabapentin, cannabis plus tricyclic anti-depressant (TCA) or cannabis plus hyaluronic acid.

This ability to combine known pain reducing properties of cannabis with a variety of different medical treatment drugs for an efficient site-specific dispensing achieved by the size adjustable implant about the cervix is unique in the art. The predetermined, prefusion transfer rate imparted by the ring material selection and orientation combined with defined placement size protocol therefore defines an advancement in pain management therapy in women's health. It will thus be seen that a new, novel and useful implantable dispensing ring for adaptable use in cannabis-based compound pain and therapeutic drug use has been illustrated and described and it will be apparent to those skilled in the art that various changes and modifications may be made therein without departing from the spirit of the invention. Therefore, I claim:

1. A method for delivering a cannabis compound to the blood stream of a female patient comprising the steps of,
A. providing an implantable dispensing permeable ring of non-biodegradable medical grade silicone,
B. placing an effective amount of cannabis-pain reduction component in said permeable ring,
C. sizing said permeable ring to fit adjustably around the cervix of a female patient,
D. inserting said adjustable fit permeable ring within the patient by a medical practitioner.

2. The method set forth in claim 1 wherein said dispensing permeable ring providing a hollow tubular member with a tapered closed end and an open access end.

3. The method set forth in claim 1 wherein said cannabis-based pain reduction is an indicia strain, a blend of cannabis indicia and Cannabis sativa.

4. The method set forth in claim 1 wherein said dispensing permeable ring forming a permeable section associated with the placement of cannabis-based pain reduction component therein.

5. The method set forth in claim 2 wherein the sizing of said permeable ring comprises, adjustably retaining said tapered closed end into said open end of said hollow tubular member.

6. The method set forth in claim 2 wherein said tapered closed end is formed of a known diameter less than that of said open tubular end.

7. The method set forth in claim 5 wherein adjustably retaining said tapered closed end in said open end comprises, providing annular ribs on the closed tapered end for registerable retainment within said open end of said tubular member.

8. The method set forth in claim 7 wherein positioning said annular ribs in longitudinal spaced relation to one another.
